(19) United States
(12) Patent Application Publication
(10) Pub. No.: US 2025/0312576 A1
(43) Pub. Date: Oct. 9, 2025

(54) HIGH-PRESSURE BALLOON

(71) Applicant: Intervention Tech, Inc., Flagstaff, AZ (US)

(72) Inventor: Michael Douglas Franklin, JR., Flagstaff, AZ (US)

(73) Assignee: Intervention Tech, Inc., Flagstaff, AZ (US)

(21) Appl. No.: 19/246,963

(22) Filed: Jun. 24, 2025

Related U.S. Application Data
(63) Continuation-in-part of application No. 18/199,941, filed on May 20, 2023.

(51) Int. Cl. A61M 25/10

(52) U.S. Cl. CPC A61M 25/1029; A61M 2025/1031; A61M 2025/1079; A61M 2025/1084; A61M 2205/32; A61M 2207/00

(57) ABSTRACT

A balloon catheter has a shaft having a Y-connector provided at the proximal end, and a balloon provided at the distal end, the balloon having a balloon body having opposing first and second ends, and a reinforcement fiber layer wrapped on the balloon body, the fiber layer formed from at least one fiber wrapped first radially around the balloon body in non-continuous rows, followed by a figure-8 wrap that continuously traverses from the first end to the second end, and the second end back to the first end.

### CROSS REFERENCE TO RELATED APPLICATIONS

This patent application is a continuation-in-part patent application claiming priority under 35 U.S.C. § 120 to U.S. patent application Ser. No. 18/199,941, entitled “HIGH-PRESSURE BALLOON FOR A CATHETER AND METHOD OF MANUFACTURE,” Filed May 20, 2023, which is incorporated by reference in its entirety for all purposes.

### TECHNICAL FIELD

The present invention relates to high-pressure balloons for catheters and methods for manufacturing the balloon.

### BACKGROUND

In surgical and transcatheter heart valves, there are different bioprosthetic implants sold with various internal designs and structures. These structures can consist of various polymers and/or metal (i.e. stainless steel, nitinol, cobalt chromium etc.) “frames” or “rings” which provide support for the other valve components such as textile sewing rings, commissure posts and tissue leaflets. In all cases, the heart valve can be expanded some amount by a high-pressure balloon to cause fracture (fracking) of the internal structures or in other cases bending/deforming some element of the structure to increase the effective overall internal diameter, so as to allow a new transcatheter heart valve to be implanted at the same location.

Existing high-pressure balloons sometimes used for these applications include the Bard True Balloon™ and Bard Atlas Gold™. These balloons can sometimes achieve the pressures needed, although their rated burst pressure (RBP) is often below the required pressure. There are other examples of fiber-reinforced high pressure balloons in use today, including the Boston Scientific Athletis™ balloon; however, these and other balloons lack the appropriate size and strength as well as other potential drawbacks.

### SUMMARY

It is an object of the present invention to provide a high-pressure balloon for use in a catheter that can be adapted to effectively frack an implanted surgical heart valve.

It is another object of the present invention to provide a balloon catheter that is intended to be used to allow needed treatment of structural heart disease patients with conditions requiring a higher-pressure balloon than existing products on the market.

To meet the objectives of the present invention, there is provided a balloon catheter having a main shaft with a Y-connector provided at a proximal end, and a balloon provided at the distal end, the balloon having a balloon body having opposing first and second ends, and a reinforcement fiber layer wrapped on the balloon body, the fiber layer including at least one continuous or non-continuous fiber wrapped radially around the balloon body, from the first end to the second end, and the second end back to the first end. The at least one fiber may be radiopaque and/or have an adhesive coating.

The balloon catheter of the present invention can be manufactured according to the following steps. First, a shaft is provided having a distal end and a proximal end, with a Y-connector provided at a proximal end, and a balloon provided at the distal end, the balloon having a balloon body having a cylindrical central section having first and second ends, a first tapered neck at the first end, a second tapered neck at the second end, a first cone at an end of the first tapered end, and a second cone at an end of the second tapered end. Next, the at least one fiber is wrapped in a radial wrap that extends from the first tapered neck and the first end across the cylindrical central section to the second end, and then traversing from the second end back to the first wrap. Next, at the end of the radial wrap, the at least one fiber is wrapped in a figure-8 wrap that traverses opposite locations of the first and second tapered neck sections, wherein the at least one fiber extends from a first location on the first tapered neck and extends across the cylindrical central section to a second location on the second tapered neck, and from the second location, extends back across the cylindrical central section to a third location on the first tapered neck that is spaced apart from the first location, and from the third location, extends back across the cylindrical central section to a fourth location on the second tapered neck that is spaced apart from the second location, and so on.

In addition to fracking surgical heart valves, other indications have been identified where the balloon according to the present invention can be beneficially used. These include but are not limited to:
1. Transcatheter Valve-in-Valve (ViV) where a previously implanted transcatheter valve has diminished functionality and it is desirable to expand it to allow a new THV (transcatheter heart valve) implantation with optimized EOA (Effective Orifice Area). These are typically aortic or pulmonary valves, but could also be mitral or tricuspid valves, depending on size.
2. Heavily calcified anatomy and structures where a hard/non-compliant ring of calcium has formed in a radial configuration requiring a non-compliant balloon catheter to avoid over-expansion. This can include native tissue valves as well as other bioprosthetics, such as pulmonary conduits, annular patches or stents implanted during prior therapies.

### BRIEF DESCRIPTION OF THE DRAWINGS

The accompanying drawings are included to provide a further understanding of the disclosure and are incorporated in and constitute a part of this specification, illustrate embodiments of the disclosure, and together with the description serve to explain the principles of the disclosure, wherein:

FIG. 1 is a schematic view of a balloon catheter according to one embodiment of the present invention.

FIG. 2A is a schematic view of the balloon of the catheter of FIG. 1 showing the first pass of the radial wrap step in the method of manufacture for the balloon.

FIG. 2B is a schematic view of the balloon of the catheter of FIG. 1 showing the second pass of the radial wrap step in the method of manufacture for the balloon.

FIG. 3A and FIG. 3B are the front views for the balloon in FIGS. 2A and 2B, respectively.

FIGS. 4A-4B are the perspective views for the balloon in FIGS. 2A and 2B, respectively.

FIG. 5A is a schematic view of the balloon of the catheter of FIG. 1 showing the first pass of the Figure-8 wrap step in the method of manufacture for the balloon.

FIG. 5B is a schematic view of the balloon of the catheter of FIG. 1 showing the second pass of the Figure-8 wrap step in the method of manufacture for the balloon.

FIG. 5C is a schematic view of the balloon of the catheter of FIG. 1 showing the third pass of the Figure-8 wrap step in the method of manufacture for the balloon.

FIGS. 6A-6C are the front views for the balloon in FIGS. 5A-5C, respectively.

FIGS. 7A-7C are the perspective views for the balloon in FIGS. 5A-5C, respectively.

FIG. 8 is an enlarged cross-sectional view of a portion of the balloon in FIG. 1.

FIG. 9 is a schematic view of a balloon with radiopaque rings on the ends of a central section of the balloon.

FIG. 10A is a schematic view of a balloon with a radiopaque fiber wrapped radially around the central section of the balloon.

FIG. 10B is a schematic view of a balloon with a radiopaque fiber wrapped transversely around the central section of the balloon.

### DETAILED DESCRIPTION OF THE ILLUSTRATED EMBODIMENTS

Persons skilled in the art will readily appreciate that various aspects of the present disclosure can be realized by any number of methods and systems configured to perform the intended functions. Stated differently, other devices, methods and systems can be incorporated herein to perform the intended functions. It should also be noted that the accompanying drawing figures referred to herein are not all drawn to scale but may be exaggerated to illustrate various aspects of the present disclosure, and in that regard, the drawing figures should not be construed as limiting. Finally, although the present disclosure can be described in connection with various principles and beliefs, the present disclosure should not be bound by theory.

In general, the present disclosure provides a high-pressure balloon that is a hybrid design between traditional blow molded medical balloons and purely constructed composite materials (i.e., the Bard True™ balloon). As a result, it incorporates strengths and benefits of both concepts by using a unique and novel method of manufacturing. Typically, when reinforcement fibers are used, they are formed using a braided, woven or knit structure formed by a machine and then transferred or directly applied to the balloon surface or other materials used in construction. The density and number of carriers of fiber for the strength generally results in an excessively large profile at the balloon cones. The present invention uses at least one strand of fiber that is wrapped in a manner that applies structural support only where primarily needed and results in sufficient radial and axial support for the pressures required.

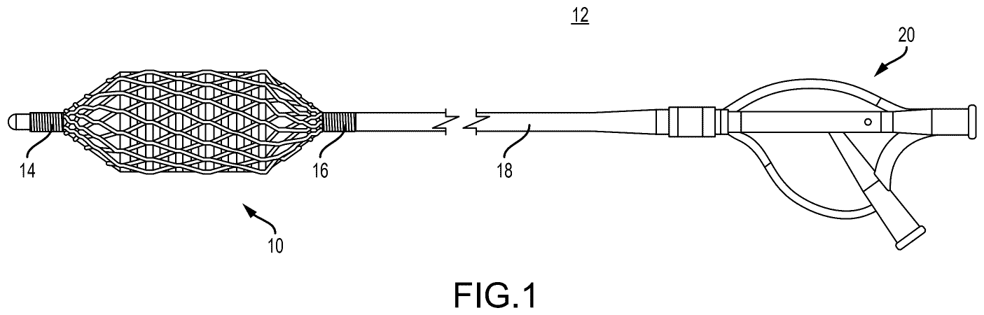

Referring to FIG. 1, the present invention provides a high-pressure balloon 10 on a catheter 12. The balloon 10 has two opposing cones 14 and 16 provided along the shaft 18 of the catheter 12. The catheter 12 includes a Y-connector 20. The construction of the catheter 12 is not critical to the balloon 10 and can be embodied in the form of any conventional over the wire or rapid exchange balloon catheter, so additional details of the catheter 12 will not be described.

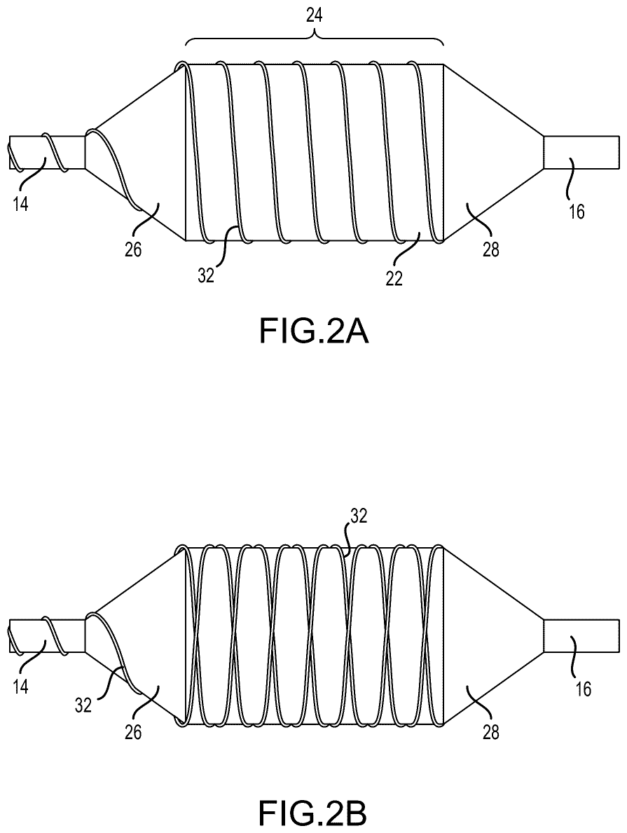

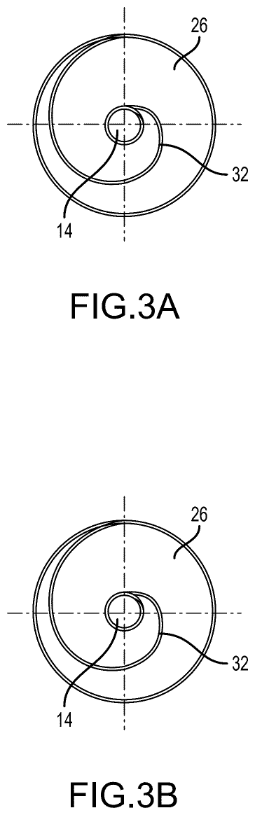

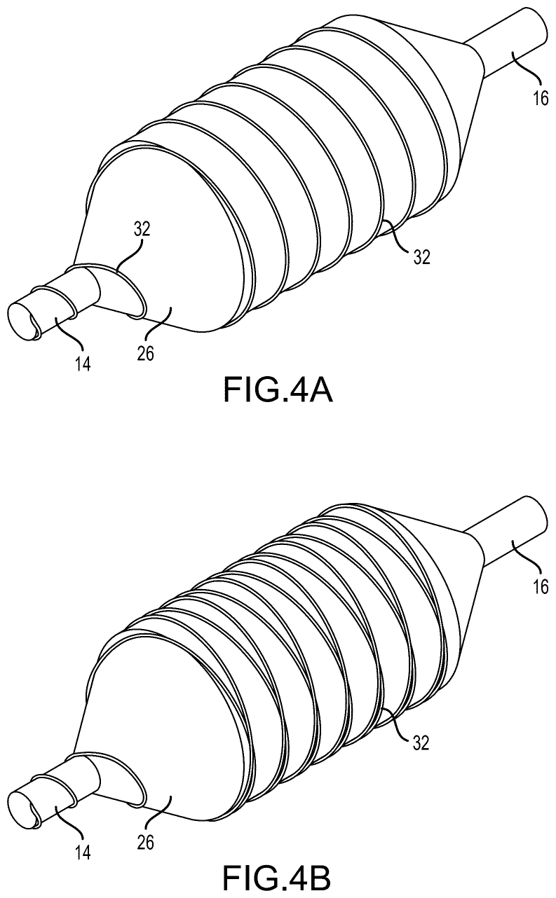

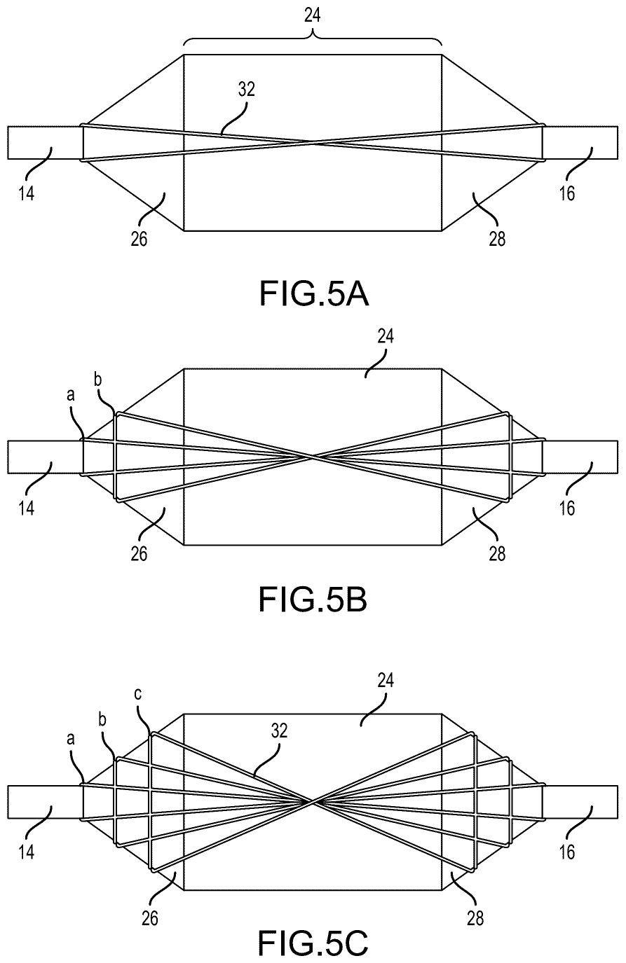

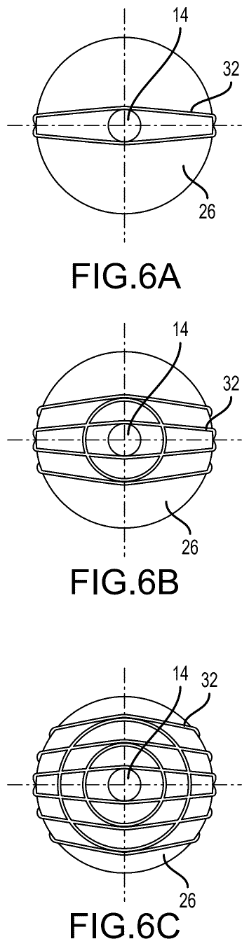

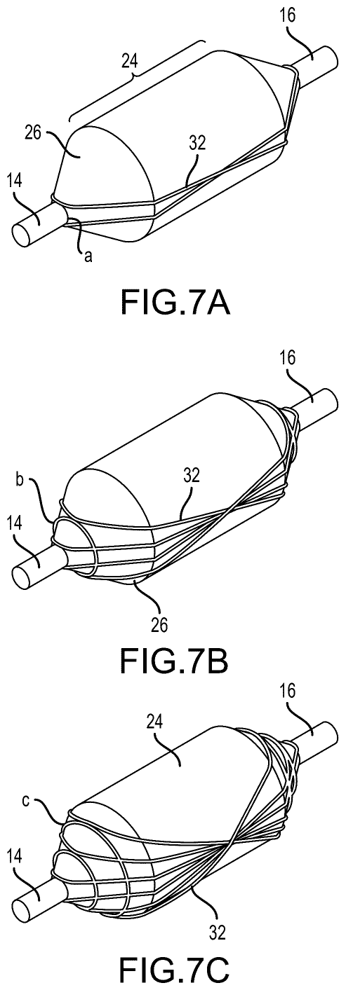

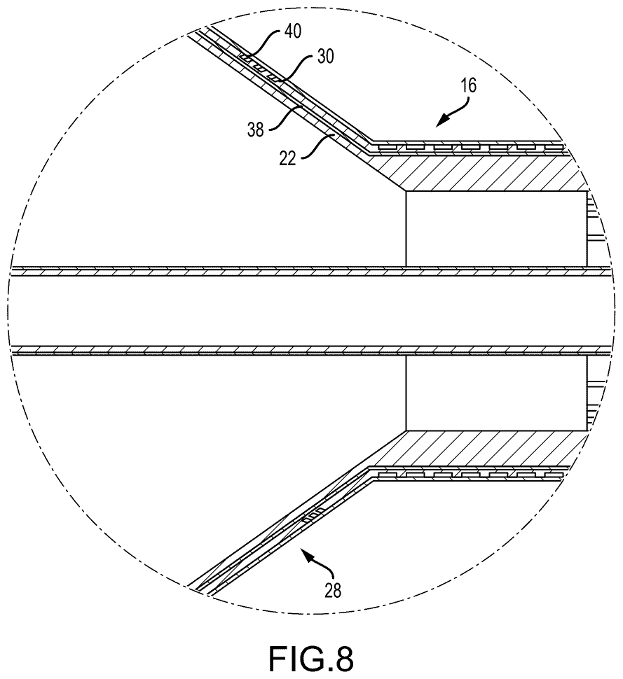

The high-pressure balloon 10 of the present invention preferably includes four primary materials/layers (see FIG. 8):
1. A conventional thin-wall blow-molded semi-compliant balloon body 22. This is currently a Nylon 12 material (e.g., Vestamid Care™ ML21) but could potentially be made from PET, PEBAX, or other medical grade polymer. This balloon body 22 can have a generally cylindrical central section 24 and opposing tapered necks 26 and 28. The balloon body 22 acts as a liner for sealing pressure and as a forming mandrel on which additional layers are formed.
2. A base coating of non-lubricious material 38 is applied to the Nylon 12 balloon. This is preferably a Kraton coating but could be another medical grade polymer. There are also surface treatments that can be performed to make the outer Nylon surface non-lubricious; such surface treatments are well-known in the art and are not described in greater detail herein. The purpose of this base coating is to make the surface of the balloon layer tacky and create the needed friction for the reinforcing fibers to stay in the appropriate locations. Alternatives using various adhesives to create the needed friction for the reinforcing fibers to stay in the appropriate locations are disclosed in detail hereinbelow.
3. A Ulteeva radial reinforcement fiber layer 30 (see FIGS. 2A-4) formed from a single continuous fiber wrapped first radially around the balloon's diameter in non-continuous rows (i.e., the space between each radial wrap that is filled in by subsequent passes in alternating directions) until adequate strength is achieved. The continuous fiber then changes pattern to a “figure-8” style axial/longitudinal support (see FIGS. 5A-7C) to an axial looping wrap which radially partial-wraps the cones and necks of the balloon while traversing from end to end continuously to reinforce the balloon's length. The reinforcing fiber layer is preferably an “Ultra High Molecular Weight Polyethylene” (UHMWPE) but can be another strong fiber. The final balloon 10 is shown in FIG. 1.
4. An encapsulating layer 40 of medical grade polymer to hold the reinforcement fiber layer together and prevent fiber migration while providing a smooth outer surface. This encapsulating layer 40 is preferably a polycarbonate-urethane material (PCU) but could be another medical grade polymer.

FIGS. 2A to 7C illustrate the method for manufacturing the balloon 10. In the first step, the base balloon body 22 is blow molded on a balloon forming machine, and then later assembled on the catheter. The initial size molds range from 19 mm to 30 mm in 1 mm increments.

In the second step, the base coating polymer (e.g., Kraton FG1901 or other) is applied to the balloon body 22 using a solvent solution dip coating process as is well-known in the art.

In the third step, the at least one reinforcement fiber layer 30 is applied using a two-axis movement. A motor spins an inflated balloon while the operator or a second motor traverses the fiber back and forth along the length of the balloon. The at least one reinforcement fiber layer 30 is applied first by a radial wrap method, and then by an axial/longitudinal figure-8 wrap method.

FIGS. 2A-4 illustrate the radial wrap method. The fiber layer 30 has a single fiber 32, though additional fibers may be used. The single fiber 32 has an initial entry path at the cone 14 and is radially wrapped around the tapered neck 26 and the central section 24 for a first pass. See FIGS. 2A, 3A and 4. The wrapping does not extend into the tapered neck 28 because radial reinforcement of both cones is not required during this step. At the end of the central section 24 adjacent the tapered neck 28, the wrapping reverses direction and returns towards the tapered neck 26 for the second pass. See FIG. 2B. On the second pass, the wrap ends at the end of the central section 24 and does not extend into the tapered neck 26. The completion of a first pass and a second pass constitutes one cycle. The radial wrapping then proceeds for another first pass and second pass to complete a second cycle. The process continues until the full radial wrap process is completed.

FIGS. 5A-7C illustrate the figure-8 wrap method. To simplify these drawings, these drawings do not show the underlying radial wrap that has already been completed. This figure-8 wrapping essentially traverses the opposite general location on the balloon body 22. This traverse is what places the lengthwise or longitudinal fiber sections into a figure-8 configuration. At the start of figure-8 wrap, the fiber 32 from the end of the radial wrap can begin from the largest-diameter section of the cone 14, which can be adjacent or immediately next to the smallest-diameter portion of the tapered neck 26. The fiber 26 traverses the tapered neck 26 across the central section 24 and the tapered neck 28, then wraps around the other cone 16 at the location where the cone 16 is adjacent or immediately next to the smallest-diameter portion of the tapered neck 28. The fiber 26 then traverses the tapered neck 28 and back across the central section 24 and the tapered neck 26. This completes a first pass as shown in FIGS. 5A, 6A and 7A. The second pass can begin at a new location along the tapered neck 26 (see FIG. 5B) and traverse to a corresponding opposite location at the tapered neck 28 before traversing back to the tapered neck 26. The third pass can begin at another new location along the tapered neck 26 (see FIG. 5C) and traverse to a corresponding opposite location at the tapered neck 28 before traversing back to the tapered neck 26. FIGS. 5A-5C show the traverse decreasing in distance along each successive pass, but it is also possible to have the figure 8 wrapping begin along the tapered necks 26 and 28 close to the central section 24 so that the traverse increases in distance along each successive pass.

In accordance with some aspects of the present disclosure, other configurations and aspects of the wrapping steps disclosed herein are contemplated. For example, where the balloon body 22 with opposing first and second ends and a reinforcement fiber layer wrapped 30 on the balloon body 22, the fiber layer may comprise at least two fibers, with at least one fiber wrapped radially around the balloon body 22, for example, in rows, and another fiber wrapped to traverse from the first end to the second end, and the second end back to the first end of the balloon body 22. The fibers may be wrapped continuously or non-continuously.

The final wrapped balloon 10 is shown in FIG. 1 and has a generally uniform distribution along the entire length and around the circumference of the balloon 10.

Although the method of the present invention provides the radial wrap before the figure-8 wrap, it is also possible to perform the figure-8 wrap before the radial wrap.

In the fourth step, the outer coating of encapsulating layer 40 of medical grade polymer is applied in the form of a PolyCarbonate-Urethane (PCU Carbothane 3585A) or similar material dip coated with a polymer/solvent solution. This layer 40 encapsulates the fiber 32 and locks the fiber wrap 30 into their positions and provides a smooth outer surface layer. The primary purpose of this layer 40 is to hold the fiber 32 in place and to provide a smooth outer coating.

As noted above, various alternatives for creating the needed friction so the reinforcing fibers stay in the appropriate locations are contemplated by the present disclosure. For example, the reinforcement fiber layers wrapped on the balloon body 22 as disclosed herein, may include a fiber with and adhesive coating applied thereto (an “adhesive fiber”) wrapped either radially, for example, in rows, around the balloon body or wrapped to traverse from the first end to the second end, and the second end back to the first end. In accordance with various aspects of the present disclosure, the adhesive fiber may comprise a single fiber or multiple fibers and may be continuous or non-continuous. In accordance with various aspects of the present disclosure, the wrapping of the fibers, may further comprise the step of applying an adhesive to the balloon body 22 prior to wrapping the adhesive fiber on the balloon body 22, after which the adhesive may be cured by any conventional or as yet unknown curing steps.

In accordance with various aspects of the present disclosure, a reinforcing fiber or fibers may be coated with an adhesive during the wrapping process. For example, the method may include providing a shaft having a distal end and a proximal end, with the balloon 10 provided at the distal end, the balloon 10 having a balloon body 22 having a central section 24 having first and second ends, a first tapered neck 26 at the first end, a second tapered neck 28 at the second end. Prior to and, for example, as the reinforcing fiber or fibers are about to be wrapped around the balloon body 22, an adhesive coating, such as a foam coating, is applied to the reinforcing fiber and then the reinforcing fiber is wrapped on the balloon body using the methods disclosed herein, as well as other undisclosed methods.

The method of the present invention is designed to provide adequate strength to the balloon, an optimized pleated/folded profile while minimizing manufacturing challenges and costs faced by more complex methods such as braiding, weaving or otherwise attaching a textile construct to augment or function as a balloon. The radial wrap is placed in non-continuous rows to prevent localized failure. This can be described as one or more sections of the fiber breaking which if continuously applied would result in the adjacent radial fibers to become loosened or weakened due to being unsupported. The figure-8 warp is designed to provide both a partial radial wrap supporting the conical neck sections while also anchoring the fiber during axial traverses to support the longitudinal section of the balloon and prevent length compliance/stretching.

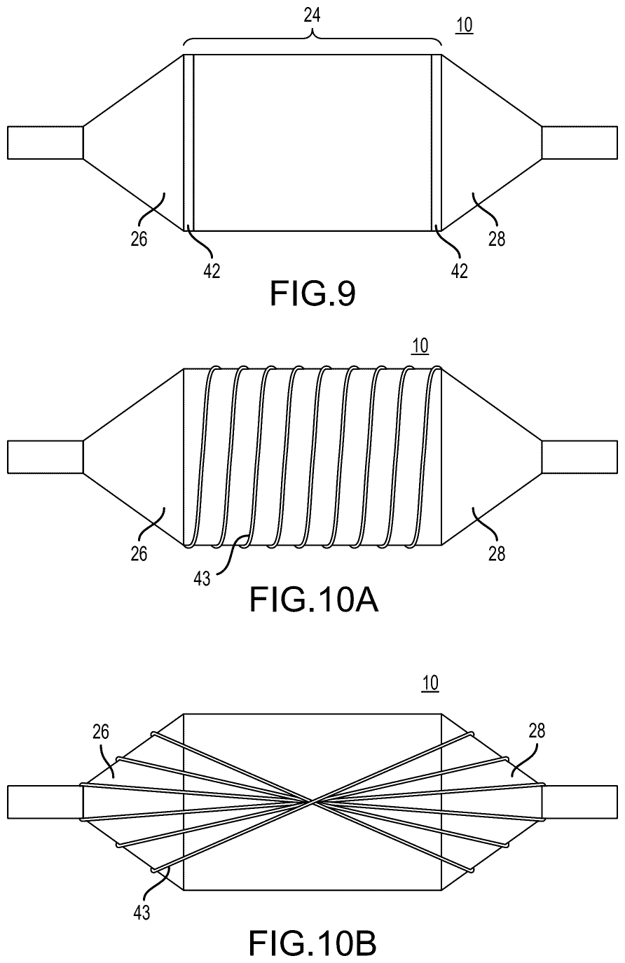

In accordance with various aspects of the present disclosure, various radiopaque characteristics and/or markers may be incorporated into the high pressure balloon 10 to improve the ability to locate the balloon 10 during use. For example, with reference to FIG. 9, one or more radiopaque rings 42 may be included on the balloon 10 at strategic locations for identifying the ends and/or a middle portion(s) of the central section 24. For example, where the balloon body 22 has a central section 24 having first and second ends, a first tapered neck 26 at the first end, a second tapered neck 28 at the second end, the radiopaque rings 42 may wrap at least partially around the balloon body 22 proximate at least one of a location where the first tapered neck 26 or the second tapered neck 28 meet the central section 24 to help identify the bounds of the central section 24.

Alternatively, a radiopaque fiber 43 may be wrapped either radially (continuously or non-continuously) around the balloon 10 (FIG. 10A) or wrapped to traverse (continuously or non-continuously) from the first end to the second end, and the second end back to the first end (FIG. 10B). In accordance with some aspects of the present disclosure, the radiopaque fiber is 43 is wrapped radially around the balloon body 22 in rows. In accordance with some aspects of the present disclosure, the radiopaque fiber 43 is wrapped radially around the balloon body 22 in rows and is also wrapped to traverse from the first end to the second end, and the second end back to the first end. In accordance with various aspects of the present disclosure, where the balloon body 22 has a central section 24 having first and second ends, a first tapered neck 26 at the first end, a second tapered neck 28 at the second end, the radiopaque fiber 43 may be wrapped primarily around the balloon body 22 proximate the locations where the first tapered neck 26 or the second tapered neck 28 meet the central section 24 to help identify the central section 24.

In accordance with some aspects of the present disclosure, the radiopaque fiber 43 may comprise a single fiber or multiple fibers, and maybe continuous or non-continuous. The radiopaque fiber 43 may comprise any now known or as yet unknown materials which provide appropriate radiopacity. For example, an exemplary radiopaque fiber 43 material includes 75D Ulteeva Purity™ radiopaque fiber. In accordance with various other aspects of the present disclosure, various radiopaque inks now known or as yet unknown may be applied to the balloon 10 instead of applying radiopaque fibers or may be used in conjunction with various radiopaque fibers, depending on the particular application.

Finally, it will be apparent to those skilled in the art that various modifications and variations can be made in the present disclosure without departing from the spirit or scope of the disclosure. Thus, it is intended that the present disclosure cover the modifications and variations of this disclosure provided they come within the scope of the appended claims and their equivalents.

Likewise, numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications may be made, especially in matters of structure, deposition materials, elements, components, shape, size, and arrangement of parts including combinations within the principles of the disclosure, to the full extent indicated by the broad, general meaning of the terms in which the appended claims are expressed. To the extent that these various modifications do not depart from the spirit and scope of the appended claims, they are intended to be encompassed therein.

I claim:

1. A balloon catheter, comprising a balloon body having opposing first and second ends, and a reinforcement fiber layer wrapped on the balloon body, the fiber layer including a radiopaque fiber wrapped either radially around the balloon body or wrapped to traverse from the first end to the second end, and the second end back to the first end.

2. The balloon catheter of claim 1, wherein the radiopaque fiber is wrapped radially around the balloon body in rows.

3. The balloon catheter of claim 1, wherein the radiopaque fiber is wrapped to continuously traverse from the first end to the second end, and the second end back to the first end.

4. The balloon catheter of claim 1, wherein the radiopaque fiber is wrapped radially around the balloon body in rows and is wrapped to continuously traverse from the first end to the second end, and the second end back to the first end.

5. The balloon catheter of claim 1, wherein the balloon body has a central section having first and second ends, a first tapered neck at the first end, a second tapered neck at the second end and the radiopaque fiber is wrapped around the balloon body proximate at least one of a location where the first tapered neck or the second tapered neck meet the central section.

6. The balloon catheter of claim 1, wherein the radiopaque fiber is a single fiber.

7. The balloon catheter of claim 1, wherein the radiopaque fiber is a continuous fiber.

8. A balloon catheter, comprising a balloon body having opposing first and second ends, and a reinforcement fiber layer wrapped on the balloon body, the fiber layer including at least two fibers, at least one fiber wrapped radially around the balloon body, and at least one fiber wrapped to traverse from the first end to the second end, and the second end back to the first end.

9. The balloon catheter of claim 8, wherein at least one fiber is wrapped radially around the balloon body in rows.

10. The balloon catheter of claim 8, wherein at least one fiber is wrapped to continuously traverse from the first end to the second end, and the second end back to the first end.

11. The balloon catheter of claim 8, wherein at least one fiber is radiopaque.

12. The balloon catheter of claim 8, wherein the radiopaque fiber is a continuous fiber.

13. A balloon catheter, comprising a balloon body having opposing first and second ends, and a reinforcement fiber layer wrapped on the balloon body, the fiber layer including an adhesive fiber wrapped either radially around the balloon body or wrapped to traverse from the first end to the second end, and the second end back to the first end.

14. The balloon catheter of claim 13, wherein the adhesive fiber is wrapped radially around the balloon body in rows.

15. The balloon catheter of claim 13, wherein the adhesive fiber is wrapped to continuously traverse from the first end to the second end, and the second end back to the first end.

16. The balloon catheter of claim 13, wherein the adhesive fiber is wrapped radially around the balloon body in rows and is wrapped to continuously traverse from the first end to the second end, and the second end back to the first end.

17. A method of making a balloon for a balloon catheter, comprising:
providing a shaft having a distal end and a proximal end, and a balloon provided at the distal end, the balloon having a balloon body having a central section having first and second ends, a first tapered neck at the first end, a second tapered neck at the second end; and
wrapping an adhesive fiber on the balloon body, the adhesive fiber wrapped either radially around the balloon body or wrapped to traverse from the first end to the second end, and the second end back to the first end.

18. The method of making a balloon for a balloon catheter of claim 17, wherein the adhesive fiber traverses between the first and second tapered neck sections, wherein the adhesive fiber extends from a first location on the first tapered neck and extends across the central section to a second location on the second tapered neck, and from the second location, extends back across the central section to a third location on the first tapered neck that is spaced apart from the first location, and from the third location, extends back across the central section to a fourth location on the second tapered neck that is spaced apart from the second location, and so on.

19. The method of making a balloon for a balloon catheter of claim 17, wherein the adhesive fiber is a single fiber.

20. The method of making a balloon for a balloon catheter of claim 17, wherein the adhesive fiber is continuous.

21. The method of making a balloon for a balloon catheter of claim 17, further comprising the step of applying an adhesive to the balloon body prior to wrapping the adhesive fiber on the balloon body.

22. The method of making a balloon for a balloon catheter of claim 17, further comprising the step of curing the adhesive fiber after wrapping the adhesive fiber on the balloon body.

23. A method of making a balloon for a balloon catheter, comprising:
providing a shaft having a distal end and a proximal end, and a balloon provided at the distal end, the balloon having a balloon body having a central section having first and second ends, a first tapered neck at the first end, a second tapered neck at the second end; and
applying an adhesive coating to a reinforcing fiber; and
wrapping the reinforcing fiber on the balloon body, the reinforcing fiber wrapped either radially around the balloon body or wrapped to traverse from the first end to the second end, and the second end back to the first end.

24. The method of making a balloon for a balloon catheter of claim 23, further comprising foam coating the adhesive on the reinforcing fiber.
